(19) United States
(12) Patent Application Publication
(10) Pub. No.: US 2024/0011997 A1
(43) Pub. Date: Jan. 11, 2024

(54) Elastin Assay

(71) Applicant: Nordic Bioscience A/S, Herlev (DK)

(72) Inventors: Karoline Natasja Stæhr Gudmann, Bronshøj (DK); Jannie Marie Bülow Sand, Måløv (US); Tina Manon-Jensen, Brøndby (DK); Diana Julie Leeming, Klampenborg (DK); Morten Karsdal, Kobenhavn Ø (DK)

(73) Assignee: Nordic Bioscience A/S, Herlev (DK)

(21) Appl. No.: 18/358,070

(22) Filed: Jul. 25, 2023

Related U.S. Application Data
(63) Continuation-in-part of application No. 16/968,277, filed on Aug. 7, 2020.

(30) Foreign Application Priority Data
Feb. 8, 2018 (EP) 1802070.1

(51) Int. Cl. G01N 33/577

(52) U.S. Cl. CPC G01N 33/577; G01N 2800/122; G01N 2800/52; G01N 2470/04

(57) ABSTRACT

Described herein are monoclonal antibodies, assay kits and immumoassay methods for quantifying elastin fragments with a C-terminus amino acid sequence LPGGYGLPYT (SEQ ID NO: 1) in a patient biofluid sample, and uses thereof for detecting or quantifying fibrotic diseases such as chronic obstructive lung disease (COPD).

### CROSS-REFERENCE TO RELATED APPLICATIONS

This is a continuation-in-part application under 35 U.S.C. § 120 of pending application U.S. Ser. No. 16/968,277, filed Aug. 7, 2020, which is a national stage application under 35 U.S.C. § 371 of international application PCT/EP2019/053003, filed Feb. 7, 2019, now abandoned, which claimed priority to foreign application GB1802070.1, filed Feb. 8, 2018, now abandoned, the entireties of all of which are hereby incorporated by reference.

### SEQUENCE LISTING

A sequence listing is electronically submitted in text format in compliance with 37 C.F.R. § 1.821(c) and is incorporated by reference herein. The XML file is named D7763CIPSEQ, was created on Jul. 21, 2023 and is 19 kb in size.

### BACKGROUND OF THE INVENTION

### Field of the Invention

The present invention relates to an elastin assay and its use in evaluating fibrotic diseases, such as COPD.

### Description of the Related Art

Chronic obstructive lung disease (COPD) is characterized by inflammation in the airways and an excessive concentration of neutrophils and mast cells (1,2). Neutrophils and mast cells synthesise and store preformed serine proteinases like cathepsin G (CG) and proteinase 3 (PR3) in granules from which they are released in response to pro-inflammatory mediators. Together these proteinases have a broad spectrum of activity against the extracellular matrix (ECM) (3,4).

Elastic fibers are a major insoluble component of the extracellular matrix of the lungs, and are essential for structure, function, resilience and elasticity of the extracellular matrix. The elastic fibers are comprised by an inner core of cross-linked elastin embedded within fibrillin microfibrils (5). They create a thin and highly branched network throughout the respiratory tree to support the expansion and recoil of the alveoli during breathing. They are characterized by a high stability, and low turnover in healthy adult tissue. Only a few proteases, such as serine proteinases in addition to selected MMPs, are able to cleave elastin fibers by damaging the elastin core and microfibrils (6-8). In a normal inflammatory response the protease-antiprotease balance is maintained through the secretion of endogenous protease inhibitors (e.g. heparan sulfate) (9). In pathological conditions this balance is skewed, which leads to a loss of elasticity, which is a major pathological feature in COPD and emphysema (10,11). It has been suggested that both the serine proteinases CG and PR3 are upregulated in COPD, but there are currently no markers that are able to link increased degradation of elastin with those proteinases.

The present inventors have now developed an immunoassay for monitoring PR3 degradation of elastin, and have demonstrated its use in evaluating fibrotic diseases, in particular COPD.

### SUMMARY OF THE INVENTION

Accordingly, in a first aspect the present invention relates to an immunoassay method for quantifying peptides having a C-terminus amino acid sequence LPGGYGLPYT (SEQ ID NO: 1) in a patient biofluid sample, said method comprising contacting said patient biofluid sample with a monoclonal antibody specifically reactive with said C-terminus amino acid sequence LPGGYGLPYT (SEQ ID NO: 1), and determining the amount of binding between said monoclonal antibody and said C-terminus amino acid sequence.

Preferably, the monoclonal antibody does not specifically recognise or bind a C-extended elongated version of said C-terminus amino acid sequence or a C-truncated shortened version of said C-terminus amino acid sequence. In this regard “C-extended elongated version of said C-terminus amino acid sequence” means one or more amino acids extending beyond the C-terminus of the sequence LPGGYGLPYT-COOH (SEQ ID NO: 1). For example, if the C-terminal amino acid sequence LPGGYGLPYT-COOH (SEQ ID NO: 1) was elongated by a threonine residue then the corresponding “C-extended elongated version” would be LPGGYGLPYTT-COOH (SEQ ID NO: 2). Similarly, “C-truncated shortened version of said C-terminus amino acid sequence” means one or more amino acids removed from the C-terminus of the sequence LPGGYGLPYT-COOH (SEQ ID NO: 1). For example, if the C-terminal amino acid sequence LPGGYGLPYT-COOH (SEQ ID NO: 1) was shortened by one amino acid residue then the corresponding “C-truncated shortened version” would be LPGGYGLPY-COOH (SEQ ID NO: 3).

The patient biofluid sample may be, but is not limited to, blood, urine, synovial fluid, serum, BALF (bronchoalveolar lavage fluid), or plasma.

The immunoassay may be, but is not limited to, a competition assay or a sandwich assay. Similarly, the immunoassay may be, but is not limited to, an enzyme-linked immunosorbent assay or a radioimmunoassay.

In a second aspect, the present invention relates to a method of immunoassay for detecting or quantifying a fibrotic disease in a patient, the method comprising contacting a patient biofluid sample with a monoclonal antibody specifically reactive with a C-terminus amino acid sequence LPGGYGLPYT (SEQ ID NO: 1), determining the amount of binding between said monoclonal antibody and peptides comprising said C-terminus amino acid sequence, and correlating said amount of binding with i) values associated with normal healthy subjects and/or ii) values associated with known fibrotic disease severity and/or iii) values obtained from said patient at a previous time point and/or iv) a predetermined cut-off value.

Preferably, the fibrotic disease is chronic obstructive pulmonary disease (COPD).

The predetermined cut-off value is preferably at least 150.0 ng/mL, more preferably at least 175.0 ng/mL, even more preferably at least 200.0 ng/mL, even more preferably at least 225.0 ng/mL, and most preferably at least 250 ng/mL. In this regard, through the combined use of various statistical analyses it has been found that a measured amount of binding between the monoclonal antibody (described above) and the C-terminus biomarker of at least 150.0 ng/mL or greater may be determinative of the presence of fibrosis, such as COPD. By having a statistical cutoff value of at least 150.0 ng/mL, more preferably at least 175.0 ng/mL, even more preferably at least 200.0 ng/mL, even more preferably at least 225.0 ng/mL, and most preferably at least 250.0 ng/mL, it is possible to utilise the method of the invention to diagnose fibrosis, such as COPD, with a high level of confidence. Or, in other words, applying the statistical cutoff value to the method of the invention is particularly advantageous as it results in a standalone diagnostic assay; i.e. it removes the need for any direct comparisons with healthy individuals and/or patients with known disease severity in order to arrive at a diagnostic conclusion. This may also be particularly advantageous when utilising the assay to evaluate patients that already have medical signs or symptoms that are generally indicative of fibrosis, such as COPD, (e.g. as determined by a physical examination and/or consultation with a medical professional) as it may act as a quick and definitive tool for corroborating the initial prognosis and thus potentially remove the need for more invasive procedures, such as endoscopy or biopsy, and expedite the commencement of a suitable treatment regimen. An expedited conclusive diagnosis may result in the disease being detected, and thus treated, at an earlier stage. Preferably, the predetermined cut-off value corresponds to the cut-off value as measured in human blood, serum or plasma.

Preferably, the monoclonal antibody does not specifically recognise or bind a C-extended elongated version of said C-terminus amino acid sequence or a C-truncated shortened version of said C-terminus amino acid sequence. The terms “C-extended” and “C-truncated” are explained supra.

The patient biofluid sample may be, but is not limited to, blood, urine, synovial fluid, serum, BALF, or plasma.

In a third aspect, the present invention relates to a method for determining whether a patient is responding positively to a treatment for a fibrotic disease, such as COPD, wherein said method comprises using the method described supra to quantify the amount of peptides comprising the C-terminus amino acid sequence LPGGYGLPYT (SEQ ID NO: 1) in at least two biological samples, said biological samples having been obtained from said patient at a first time point and at at least one subsequent time point during a period of administration of the treatment to said patient, and wherein a reduction in the quantity of peptides comprising the C-terminus amino acid sequence LPGGYGLPYT (SE ID NO: 1) from said first time point to said at least one subsequent time point during the period of treatment is indicative of said patient responding positively to said treatment.

The above method may also be used to determine the efficacy of a novel therapeutic for treating a fibrotic disease, such as COPD. In that regard, a novel therapeutic will be considered efficacious if the quantity of peptides comprising the C-terminus amino acid sequence LPGGYGLPYT (SEQ ID NO: 1) is reduced from the said first time point to said at least one subsequent time point during the period of treatment using said novel therapeutic.

In a fourth aspect, the present invention relates to a monoclonal antibody specifically reactive with a C-terminus amino acid sequence LPGGYGLPYT (SEQ ID NO: 1). The term “monoclonal antibody” as used herein extends to intact monoclonal antibodies and antibody fragments thereof, such as F_{ab}, Fᵥ, etc., that are specifically reactive with a C-terminus amino acid sequence LPGGYGLPYT (SEQ ID NO: 1).

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity. Antibodies which retain the same binding specificity may contain the same complementarity-determining regions (CDR). The CDR of an antibody can be determined using methods know in the art such as that described by Kabat et al.

Antibodies can be generated from B cell clones as described in the examples. The isotype of the antibody can be determined by ELISA specific for human IgM, IgG or IgA isotype, or human IgG1, IgG2, IgG3 or IgG4 subclasses. Other suitable methods can be used to identify the isotype. In the present invention, the monoclonal antibody may comprise any constant region known in the art. Human constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4. The monoclonal antibody may preferably be of the IgG isotype, including any one of IgGI, IgG2, IgG3 or IgG4.

The amino acid sequence of the antibodies generated can be determined using standard techniques. For example RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

Monoclonal antibodies that specifically bind to the C-terminus amino acid sequence LPGGYGLPYT-COOH (SEQ ID NO: 1) can be generated via any suitable techniques known in the art. For example, the monoclonal antibody may be raised against a synthetic peptide having the amino acid sequence LPGGYGLPYT (SEQ ID NO: 1), such as for example by: immunizing a rodent (or other suitable mammal) with a synthetic peptide consisting of the sequence LPGGYGLPYT (SEQ ID NO: 1), which optionally may linked to an immunogenic carrier protein (such as keyhole limpet hemocyanin), isolating and cloning a single antibody producing cell, and assaying the resulting monoclonal antibodies to ensure that they have the desired specificity. An exemplary protocol for producing a monoclonal antibody that that specifically bind to the N-terminus amino acid sequence LPGGYGLPYT (SEQ ID NO: 1) is described infra.

Preferably, the monoclonal antibody or fragment thereof may preferably comprise one or more complementarity-determining regions (CDRs) selected from:

CDR-L1:
(SEQ ID NO: 8)
HASQNINVWLS
CDR-L2:
(SEQ ID NO: 9)
KASNLHT
CDR-L3:
(SEQ ID NO: 10)
QQGQSYPLT
CDR-H1:
(SEQ ID NO: 11)
DYYMS
CDR-H2:
(SEQ ID NO: 12)
FIRNKANGYTTEYSASVKG
CDR-H3:
(SEQ ID NO: 13)
DIITTPSWFGY

Preferably the antibody or fragment thereof comprises at least 2, 3, 4, 5 or 6 of the above listed CDR sequences.

Preferably the monoclonal antibody or fragment thereof has a light chain variable region comprising the CDR sequences:

CDR-L1:
(SEQ ID NO: 8)
HASQNINVWLS
CDR-L2:
(SEQ ID NO: 9)
KASNLHT
CDR-L3:
(SEQ ID NO: 10)
QQGQSYPLT

Preferably the monoclonal antibody or fragment thereof has a light chain that comprises framework sequences between the CDRs, wherein said framework sequences are substantially identical or substantially similar to the framework sequences between the CDRs in the light chain sequence below (in which the CDRs are shown in bold, and the framework sequences are shown in italics):

### HASQNINVWLSWYQQKPGNIPKLLIYKASNLHTGVPSRFSGSGSGTGFTLTISSLOPEDIAT YYCQQGQSYPLT (SEQ ID NO: 14).

Preferably the monoclonal antibody or fragment thereof has a heavy chain variable region comprising the CDR sequences:

CDR-H1:
(SEQ ID NO: 11)
DYYMS
CDR-H2:
(SEQ ID NO: 12)
FIRNKANGYTTEYSASVKG
CDR-H3:
(SEQ ID NO: 13)
DIITTPSWFGY

Preferably the monoclonal antibody or fragment thereof has a heavy chain that comprises framework sequences between the CDRs, wherein said framework sequences are substantially identical or substantially similar to the framework sequences between the CDRs in the heavy chain sequence below (in which the CDRs are shown in bold, and the framework sequences are shown in italics):

(SEQ ID NO: 15)
DYYMSWVRQPPGKALEWLGFIRNKANGYTTEYSASVKGRFTISRDN
SQSILYLQMNTLRAEDSATYYCARDIITTPSWFGY.

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similarity or identity may be measured over the entire length of each intervening framework sequence. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody or fragment thereof may comprise the light chain variable region sequence:

(SEQ ID NO: 16)
DIQMNQSPSSLSASLGDTITITCHASQNINVWLSWYQQKPGNIPKL
LIYKASNLHTGVPSRFSGSGSGTGFTLTISSLQPEDIATYYCQQGQ
SYPLTFGAGTKLELK
(CDRs bold; Framework sequences in italics)
and/or
the heavy chain variable region sequence:
(SEQ ID NO: 17)
EVKLVESGGGLVQPGDSLRLSCATSGFTFTDYYMSWVRQPPGKALE
WLGFIRNKANGYTTEYSASVKGRFTISRDNSQSILYLQMNTLRAED
SATYYCARDIITTPSWFGYWGQGTLVTVSA
(CDRs bold; Framework sequences in italics).

In a fifth aspect, the present invention relates to an assay kit comprising a monoclonal antibody specifically reactive with a C-terminus amino acid sequence LPGGYGLPYT (SEQ ID NO: 1), and at least one of:
a streptavidin coated well plate,
a biotinylated peptide Biotin-L-LPGGYGLPYT (SEQ ID NO: 5), wherein L is an optional linker,
a secondary antibody for use in a sandwich immunoassay,
a calibrator peptide comprising the C-terminus amino acid sequence LPGGYGLPYT (SEQ ID NO: 1),
an antibody biotinylation kit,
an antibody HRP labeling kit,
an antibody radiolabeling kit.

Preferably, the monoclonal antibody of the invention is raised against a synthetic peptide having the amino acid sequence LPGGYGLPYT (SEQ ID NO: 1).

### Definitions

As used herein the term “C-terminus” refers to the extremity of a polypeptide, i.e. at the C-terminal end of the polypeptide, and is not to be construed as meaning in the general direction thereof.

As used herein the term “competitive ELISA” refers to a competitive enzyme-linked immunosorbent assay. In a “competitive ELISA” the target peptide present in a sample (if any) competes with a known amount of target of peptide (which for example is bound to a fixed substrate or is labelled) for to binding an antibody, and is a technique known to the person skilled in the art.

As used herein the term “sandwich immunoassay” refers to the use of at least two antibodies for the detection of an antigen in a sample, and is a technique known to the person skilled in the art.

As used herein the term “amount of binding” refers to the quantification of binding between antibody and biomarker, which said quantification is determined by comparing the measured values of biomarker in the biofluid samples against a calibration curve, wherein the calibration curve is produced using standard samples of known concentration of the biomarker. In the specific assay disclosed herein which measures in biofluids the C-terminus biomarker having the C-terminus amino acid sequence LPGGYGLPYT (SEQ ID NO: 1), the calibration curve is produced using standard samples of known concentration of the calibration peptide LPGGYGLPYT (SEQ ID NO: 1). The values measured in the biofluid samples are compared to the calibration curve to determine the actual quantity of biomarker in the sample. The present invention utilises spectrophotometric analysis to both produce the standard curve and measure the amount of binding in the biofluid samples; in the Examples set out below the method utilises HRP and TMB to produce a measurable colour intensity which is proportional to the amount of binding and which can be read by the spectrophotometer. Of course, any other suitable analytical method could also be used.

The term “specifically bind” as used herein means that the antibody binding is selective for the antigen and that this binding can be distinguished from unwanted or non-specific interactions. The ability of a monoclonal antibody to bind to a specific epitope or peptide sequence can be measured either through an enzyme-linked immunosorbent assay (ELISA) as described herein or other techniques familiar to one of skill in the art, e.g. surface plasmon resonance (SPR) technique (analyzed e.g. on a BIAcore instrument) and traditional binding assays. The extent of binding of a monoclonal antibody to an unrelated protein is less than about 10% of the binding of the monoclonal antibody to the epitope or peptide as measured, e.g., by ELISA. “Affinity” refers to the strength of the sum total of non-covalent interactions between a single binding site of a molecule (e.g., an epitope binding region of an antibody) and its binding partner (e.g., an epitope or antigen). Unless indicated otherwise, as used herein, “binding affinity” refers to intrinsic binding affinity which reflects a 1:1 interaction between members of a binding pair (e.g., an antigen binding moiety and an antigen). The affinity of a molecule for its partner can generally be represented by the dissociation constant (Kd), which is the ratio of dissociation and association rate constants (Koff and Kon, respectively). Thus, equivalent affinities may comprise different rate constants, as long as the ratio of the rate constants remains the same. The dissociation constant represents the concentration of the antigen at which half of the binding sites on the antibody are occupied. A lower Kd indicates a higher binding affinity between the antibody and antigen, while a higher Kd reflects weaker binding. Several methods are available to measure the Kd of an antibody, including surface plasmon resonance (SPR), isothermal titration calorimetry (ITC), and fluorescence-based assays. In certain aspects, a monoclonal antibody that binds to the epitope or peptide has a dissociation constant (Kd) of <1 pM, <100 nM, <10 nM, <1 nM, <nM, <0.01 nM, or <0.001 nM (e.g. 10⁸ M or less, e.g. from 10⁸ M to 10¹³ M, e.g., from 10⁹ M to 10¹³ M).

As used herein the “cut-off value” means an amount of binding that is determined statistically to be indicative of a high likelihood of a fibrotic disease, such as COPD, in a patient, in that a measured value of biomarker in a patient sample that is at or above the statistical cut-off value corresponds to at least a 70% probability, preferably at least an 80% probability, preferably at least an 85% probability, more preferably at least a 90% probability, and most preferably at least a 95% probability of the presence or likelihood of a fibrotic disease, such as COPD.

As used herein the term “values associated with normal healthy subjects and/or values associated with known disease severity” means standardised quantities of ELP-3 determined by the method described supra for subjects considered to be healthy, i.e. without a fibrotic disease, such as COPD, and/or standardised quantities of ELP-3 determined by the method described supra for subjects known to have a fibrotic disease, such as COPD, of a known severity.

As used herein, “ELP-3” refers to peptides having the C-terminus amino acid sequence LPGGYGLPYT (SEQ ID NO: 1), and “ELP-3 ELISA” refers to the specific competitive ELISA disclosed herein which quantifies in a sample the amount peptides having the C-terminus amino acid sequence LPGGYGLPYT (SEQ ID NO: 1).

### BRIEF DESCRIPTION OF THE DRAWINGS

FIG. 1. Measurement of ELP-3 in healthy subjects and in patients with COPD.

### DETAILED DESCRIPTION OF THE INVENTION

### Examples

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.

### Reagents

All applied reagents used in the presented experiments were high quality chemicals from Sigma Aldrich (St. Louis, Mo, USA) and Merck (Whitehouse Station, NJ, USA). The 96-well streptavidin-coated microtiterplates used were from Roche, Basel, Switzerland. The assay buffer applied consisted of 50 mM Tris-buffered saline (TBS) 2 g NaCl, pH 8.0. 3,3′,5,5′-Tetramethylbenzidine (TMB) was from Kem-EN-Tec Diagnostics. The synthetic peptides used for immunization and assay development were 1) Immunogenic peptide: KLH-CGG-LPGGYGLPYT (SEQ ID NO: 4), Biotinylated peptide: Biotin-LPGGYGLPYT (SEQ ID NO: 5), 3) Standard peptide: LPGGYGLPYT (SEQ ID NO: 1), and 4) Elongated peptide: LPGGYGLPYTT (SEQ ID NO: 2). All synthetic peptides were purchased from American peptide Company, Sunnyvale, California USA. Reagents applied for in vitro cleavages: Elastin (Sigma, E7152) Human neutrophil elastase (HNE) protein (Abcam, ab91099), PR3 (EPC ML734), and complete protease inhibitor (Roche 1186153001).

### Selection of Target Sequence

Cleavage sites specific for PR3 in elastin (Uniprot: P15502—ELN_Human) have previously been identified by mass spectrometry (12). The PR3 generated elastin decapeptides were blasted for homology to decapeptide sequences from other proteins using the NPS@: network protein sequence analysis. Based on this the sequence LPGGYGLPYT (SEQ ID NO: 1) was selected for the ELP-3 assay antibody development. The presence of the identified sequence was validated by mass spectrometry in-house analysis of PR3 cleavage of elastin in vitro.

### Antibody Development

Immunizations were performed in 6 mice, for each selected target sequence, that were 4-6 weeks old using Freund's incomplete adjuvant (KLH-CGG-LPGGYGLPYT (SEQ ID NO: 4)) subcutaneously in the abdomen with 200 ul emulsified antigen (50 ug per immunization). The four initial immunizations were performed every second week followed by additional four immunizations performed every fourth week. The mouse with the highest serum titer was selected for fusion. The mouse was rested for a month and then boosted intravenously with 50 ul immunogenic peptide in 100 ul 0.9% NaCl solution three days before isolation of the spleen. The spleen cells were fused with SP2/o myeloma cells to produce hybridroma as described by Gefter et al. and cloned in culture dishes (13). The clones were plated into 96-well microtiter plates for further growth employing the limited dilution method to ensure monoclonal growth. Supernatants of antibody-producing hybridoma were screened for reactivity against standard peptide and native material in an indirect ELISA using streptavidin-coated plates. Biotin-LPGGYGLPYT (SEQ ID NO: 5) was used as screening peptide, while standard peptide LPGGYGLPYT (SEQ ID NO: 1) was used as a calibrator to test for further specificity of clones.

Clones were selected by testing the antibodies' selective and unique reactivity towards the selection peptide or the immunogenic peptide. The selected antibody was isotyped using the SBA Clonotyping™ System-HRP (Southern Biotech, Birmingham, AL, USA), and purified using a protein-G column from GE healthcare Life Sciences (Little Chalfont, Buckinghamshire, UK).

The selected antibody was sequenced and the CDRs determined. Total RNA was isolated from the hybridoma cells following the technical manual of Zymogen Quick-RNA Miniprep Kit. Total RNA was then reverse transcribed into cDNA using isotype-specific anti-sense primers or universal primers following the technical manual of SMARTScribe™ Reverse Transcriptase Kit. The antibody fragments of VH and VL were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence of these clones was provided.

The sequence of the chains are as follows (CDRs in bold; Framework sequence in Italics; Constant region underlined):

Light chain: Amino acid sequence (127 aa)
(SEQ ID NO: 18)
DIQMNQSPSSLSASLGDTITITCHASQNINVWLSWYQQKPGNIPKL
LIYKASNLHTGVPSRFSGSGSGTGFTLTISSLQPEDIATYYCQQGQ
SYPLTFGAGTKLELKRADAAPTVSIFPPSSEQLTSGGASVVCFLNN
FYPKDINVKWKIDGSERQNGVLNSWTDQDSKDSTYSMSSTLTLTKD
EYERHNSYTCEATHKTSTSPIVKSFNRNEC
CDR-L1:
(SEQ ID NO: 8)
HASQNINVWLS
CDR-L2:
(SEQ ID NO: 9)
KASNLHT
CDR-L3:
(SEQ ID NO: 10)
QQGQSYPLT
and
Heavy chain: Amino acid sequence (141 aa):
(SEQ ID NO: 19)
EVKLVESGGGLVQPGDSLRLSCATSGFTFTDYYMSWVRQPPGKALE
WLGFIRNKANGYTTEYSASVKGRFTISRDNSQSILYLQMNTLRAED
SATYYCARDIITTPSWFGYWGQGTLVTVSAAKTTPPSVYPLAPGSA
AQTNSMVTLGCLVKGYFPEPVTVTWNSGSLSSGVHTFPAVLQSDLY
TLSSSVTVPSSTWPSETVTCNVAHPASSTKVDKKIVPRDCGCKPCI
CTVPEVSSVFIFPPKPKDVLTITLTPKVTCVVVDISKDDPEVQFSW
FVDDVEVHTAQTQPREEQFNSTFRSVSELPIMHQDWLNGKEFKCRV
NSAAFPAPIEKTISKTKGRPKAPQVYTIPPPKEQMAKDKVSLTCMI
TDFFPEDITVEWQWNGQPAENYKNTQPIMDTDGSYFVYSKLNVQKS
NWEAGNTFTCSVLHEGLHNHHTEKSLSHSPGK
CDR-H1:
(SEQ ID NO: 11)
DYYMS
CDR-H2:
(SEQ ID NO: 12)
FIRNKANGYTTEYSASVKG
CDR-H3:
(SEQ ID NO: 13)
DIITTPSWFGY

### ELISA Protocol

Supernatant from a selected clone for the targeted sequence was collected and monoclonal antibody was purified using HiTrap affinity columns (GE Healthcare Life Science, Little Chalfront, Buckinghamshire, UK).

Based on the collected antibodies the ELP-3 assay was developed as competitive ELISA assay. A 96-well streptavidin-coated microtiter plate (Roche, Basel, Switzerland) was coated with 100 ul biotinylated screening peptide (Biotin-LPGGYGLPYT (SEQ ID NO: 5)) dissolved in assay buffer for 30 min at 20° C. with shaking. Plates were washed five times in washing buffer (20 mM TRIS, 50 mM NaCl, pH 7). Sample/standard/control (20 ul) was added and followed by addition of (100 ul) monoclonal antibody and incubated for 3 h at 4° C. with shaking. After incubation, plates were washed five times in washing buffer. A volume of 100 ul of secondary HRP labeled goat anti-mouse antibody was added followed by 1 h incubation at 20° C. with shaking. Plates were then washed and TMB was added and the plate was analyzed by a SpectraMax M reader (Molecular Devices, Ca, USA) at 450 nm with 650 nm as the reference. A standard curve was plotted using a 4-parametric mathematical fit model. Each ELISA plate included kit controls to monitor inter-assay variation. All samples were measured within the range of the assay. All samples below the level of lower limit of quantification (LLOQ) were assigned the value of LLOQ.

### Technical Evaluation

To determine linearity of dilution healthy human serum (n=4), heparin plasma (n=4), citrate plasma (n=4) and EDTA plasma was applied. Antibody specificity was calculated as percentage of signal inhibition of 2-fold diluted standard peptide (LPGGYGLPYT (SEQ ID NO: 1)), elongated peptide (LPGGYGLPYTT (SEQ ID NO: 2)), non-sense peptide (PGGVPGGVFY (SEQ ID NO: 6)) and non-sense coater (LPGGYGLPYT-K-Biotin (SEQ ID NO: 7)). The linarites were assessed by the percentage recovery of undiluted samples. The intra and inter-assay variation was determined by 10 independent measurements of 7 quality control samples run in double determinations. Lower limit of detection (LLOD) was determined as mean+3× standard deviations (SD) of 21 blank (buffer) samples. Upper limit of detection (ULOD) was determined as the mean −3×SD measurements of standard A. Lower limit of quantification (LLOQ) was determined as the lowest concentration measured in human serum with an error lower than 30%. The accuracy was measured in healthy human serum samples spiked with standard peptide and in serum, and then calculated as the percentage recovery of spiked peptide or serum in buffer. The assay was tested for interference by calculating the percentage recovery of analyte in non-spiked healthy serum compared to human serum spiked with lipids (low=4.83 mM, high=10.98 mM), hemoglobin (low=0.155 mM, high=0.310 mM), and biotin (low=30 ng/ml, high=90 ng/ml).

### Analyte Stability

Analyte stability was evaluated by incubation of three healthy human serum and plasma samples at either 4 or 20° C. for 2, 4 and 24 hours and calculated as the percentage of the samples kept at −20 C (0 hour sample). Additionally, the freeze thaw stability was determined for three healthy human serum and plasma samples to up to four freeze and thaw.

### Cleavage Analysis

In order to generate the ELP-3 fragment in vitro, elastin was incubated for 2, 4, 24 or 48 hours at 37° C. with PR3 or HNE. Elastin was reconstituted in buffer (Tris-HCl, 150 mM NaCl, pH 7.5) for a concentration of 1 mg/ml. Cleavage solutions contained a final concentration of 100 ug/ml elastin and 1 ug/ml protease for the PR3 and 100 ug/ml elastin and 2 ug/ml protease for the HNE cleavage (buffer: Tris-HCl, 150 mM NaCl, pH 7.5). All solutions were immediately frozen until analysis.

### Ethics Statement

The work performed in mice was approved by the National Authority (The Animal Experiments inspectorate, approval number: 2013-15-2934-00956). All mice were treated according to the guidelines for animal welfare.

The assessment of ELP-3 in COPD patients was performed in a cohort previously described by Sand et al. (14). The study included 68 participants and complies with the Declaration of Helsinki and Good clinical practice Guidelines, was approved by the local ethics committee (protocol number H-6-2013-014). All participants provided informed consent before all study-related assessments.

Inclusion criteria were a COPD diagnosis and FEV1<80% of predicted value.

Exclusion criterion was an acute exacerbation of COPD leading to hospitalization within four weeks prior to the study. ELP-3 levels in the COPD patients were compared to the levels of commercially available control sera from healthy donors (Valley Bio-Medial, Winchester, VA, USA). All measurements were performed blinded.

### Statistical Analyses

Differences in ELP-3 levels between healthy controls and COPD patients were assed using a two-tailed Mann-Whitney unpaired t-test. Comparison of age in healthy controls vs COPD patients was performed using the Mann-Whitney unpaired t-test. Differences were considered statistically significant if p<0.05. Asterisks indicate the following: *p<0.05, **p<0.01, ***p<0.001, ****p<0.0001. Graphs are shown as Mean±Standard error of the mean. All statistical analyses were performed in GraphPad Prism v. 7 (Graph Pad Software, La Jolla, CA, USA) or MedCalc (Ostend, Belgium).

### Results

### Clone Characterization

Supernatants from antibody producing hybridomas were screened for reactivity against standard peptide, elongated peptide, and truncated peptide in an indirect ELISA. The peptide Biotin-LPGGYGLPYT (SEQ ID NO: 5) was used to screen for reactivity. The antibody clone NBH116/6A10-E7-D12 was selected for ELP-3 and antibodies were purified from the supernatant. Based on this the competitive ELISA assay was established. Native reactivity was observed in serum and plasma, and no inhibition was observed using the elongated peptide or non-sense peptide.

### Technical Evaluation

The measurement was determined based on the linear range of the standard curve. The mean intra- and inter-assay variation ranged from 2.4-10% and 4.7-14.5%, respectively.

Linearity of dilution was observed when diluted 1+3 for human serum and citrate plasma (but not EDTA and heparin plasma). Serum samples were spiked with peptide or another serum sample in seven different concentrations. The spiking recovery for both serum and peptides were within an acceptable range. Mean recovery % values were within ±20% in reference with expected concentration of spiked samples and therefore acceptable. Analyte stability was acceptable during short term storage (up to 48 hours) at 4 and 20° C., during freeze/cycles and neither hemoglobin, lipids or biotin reduced the signal for any of the two assays.

### In Vitro Analysis—ELP-3 is Generated by PR3

Elastin was incubated with different elastases in order to determine whether ELP-3 was specific for proteinase 3 cleavage. ELP-3 was generated by elastin cleaved by PR3 after 2-48 h. Detectable levels of fragments recognized by ELP-3 were generated by HNE cleavage, but in much lower concentrations compared to the respective assay. No cross reactivity was observed towards the intact elastin, buffer control or protease controls for the assay.

ELP-3 Levels were Increased in COPD as Compared to Healthy Controls

To determine whether ELP-3 levels are increased in COPD, ELP-3 was assessed in a previously described cohort of 68 patients with clinically stable COPD. Of these, 26 also attended a follow-up visit after four weeks. Twenty two samples from healthy donors were included for comparison. Demographics can be seen in Table 1 and have previously been described by Sand et al. (14).

TABLE 1
Patient demographics. Mean (SD)

| | COPD | | Healthy control | |
|---|---|---|---|---|
| | (n = 68) | | (n = 26) | p-value |
| Age, Mean | 71.1 | (9.0) | | <0.0001 |
| (SD) | | | | |
| Male, n (%) | 40 | (59) | | 0.52 |
| BMI, Mean | 24.5 | (6.1) | NA | — |
| (SD) | | | | |
| FEV1% of | 40.4 | (16.3) | NA | — |
| predicted | | | | |
| value | | | | |
| FEV/FVC | 49.6 | (0.14) | NA | — |
| ratio % | | | | |
| GOLD 2/3/4, | 34%/34%/32% | | NA | — |
| % | | | | |
| ELP-3 (ng/ml), | 217.9 | (168.6-255.0) | 38.4 (33.8-42.3) | p < 0.0001 |
| Median | | | | |
| (95% CI) | | | | |

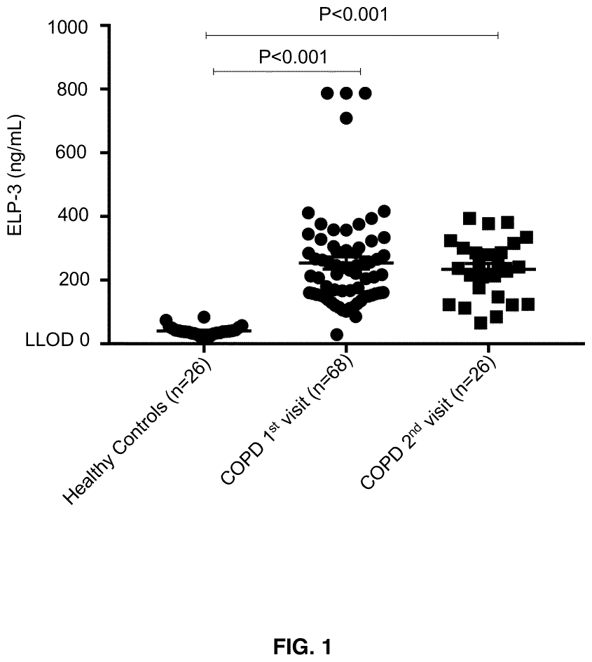

As demonstrated in FIG. 1, ELP-3 is statistically elevated in COPD patients when compared to healthy subjects (p<0.0001).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference.

### REFERENCES

1. Barnes et al., Nat Rev Dis Prim, 2015:15076.
2. Decramer et al., Lancet, 379:1341-1351, 2012.
3. Owen, Int J Chron Obstruct Pulmon Dis, 3:253-268, 2008.
4. Barnes et al., Pharmacol Rev, 2004, 56:515, 20024.
5. Baldwin et al., Expert Rev Mol Med, 2013, 15:e8, 2013.
6. Kielty et al., FEBS Lett, 1994, 351:85-89, 1994.
7. Ashworth et al I., Biochem J, 340 (Pt 1:171-181), 1999.
8. Petersen et al., Eur J Vasc Endovasc Surg, 22:48-52, 2001.
9. Craciun et al., Glycobiology, 26:701-709, 2016.
10. Dillon et al. I., Am Rev Respir Dis, 146:1143-1148, 1992.
11. Schriver et al., Am Rev Respir Dis, 145:762-766, 1992.
12. Heinz et al., Biochimie, 94:192-202, 2012.
13. Gefter et al., Somatic Cell Genet, 1977, 3:231-236, 1977.
14. Sand et al., Respir Res, 16:69, 2015.
tive lung disease (COPD).

What is claimed is:

1. An immunoassay method for quantifying peptides having a C-terminus amino acid sequence LPGGYGLPYT (SEQ ID NO: 1) in a patient biofluid sample, said method comprising contacting said patient biofluid sample with a monoclonal antibody specifically reactive with said C-terminus amino acid sequence LPGGYGLPYT (SEQ ID NO: 1), and determining the amount of binding between said monoclonal antibody and said C-terminus amino acid sequence.

2. The method of claim 1, wherein the monoclonal antibody does not specifically recognise or bind a C-extended elongated version of said C-terminus amino acid sequence or a C-truncated shortened version of said C-terminus amino acid sequence.

3. The method of claim 1, wherein the patient biofluid sample is blood, urine, synovial fluid, serum, BALF, or plasma.

4. A method of immunoassay for detecting or quantifying a fibrotic disease in a patient, the method comprising contacting a patient biofluid sample with a monoclonal antibody specifically reactive with a C-terminus amino acid sequence LPGGYGLPYT (SEQ ID NO: 1), determining the amount of binding between said monoclonal antibody and peptides comprising said C-terminus amino acid sequence, and correlating said amount of binding with i) values associated with normal healthy subjects and/or ii) values associated with known fibrotic disease severity and/or iii) values obtained from said patient at a previous time point and/or iv) a predetermined cut-off value.

5. A method as claimed in claim 4, wherein the fibrotic disease is chronic obstructive pulmonary disease (COPD).

6. A method as claimed in claim 4, wherein the predetermined cut-off value is at least 150.0 ng/mL.

7. The method of claim 4, wherein the monoclonal antibody does not specifically recognise or bind a C-extended elongated version of said C-terminus amino acid sequence or a C-truncated shortened version of said C-terminus amino acid sequence.

8. The method of claim 4, wherein the patient biofluid sample is blood, urine, synovial fluid, serum, BALF, or plasma.

9. A method for determining whether a patient is responding positively to a treatment for a fibrotic disease, wherein said method comprises using the method of claim 1 to quantify the amount of peptides comprising the C-terminus amino acid sequence LPGGYGLPYT (SEQ ID NO: 1) in at least two patient biofluid samples, said patient biofluid samples having been obtained from said patient at a first time point and at at least one subsequent time point during a period of administration of the treatment to said patient, and wherein a reduction in the quantity of peptides comprising the C-terminus amino acid sequence LPGGYGLPYT (SEQ ID NO: 1) from said first time point to said at least one subsequent time point during the period of treatment is indicative of said patient responding positively to said treatment.

10. The method of claim 9, wherein the fibrotic disease is COPD.

11. A monoclonal antibody specifically reactive with a C-terminus amino acid sequence LPGGYGLPYT (SEQ ID NO: 1).

12. An assay kit comprising a monoclonal antibody specifically reactive with a C-terminus amino acid sequence LPGGYGLPYT (SEQ ID NO: 1), and at least one of:
a streptavidin coated well plate;
a biotinylated peptide Biotin-L-LPGGYGLPYT (SEQ ID NO: 5), wherein L is an optional linker;
a secondary antibody for use in a sandwich immunoassay;
a calibrator peptide comprising the C-terminus amino acid sequence LPGGYGLPYT (SEQ ID NO: 1) an antibody biotinylation kit;
an antibody HRP labeling kit; or
an antibody radiolabeling kit.

13. An assay kit as claimed in claim 12, wherein the monoclonal antibody is raised against a synthetic peptide having the amino acid sequence LPGGYGLPYT (SEQ ID NO: 1).


SEQUENCE LISTING (19 sequences)

SEQ ID NO: 1 - protein, 10 aa, Homo sapiens
LPGGYGLPYT

SEQ ID NO: 2 - protein, 11 aa, Homo sapiens
LPGGYGLPYTT

SEQ ID NO: 3 - protein, 9 aa, Homo sapiens
LPGGYGLPY

SEQ ID NO: 4 - protein, 13 aa, synthetic construct
  MOD_RES 1: note = Bound to keyhole limpet hemocyanin
CGGLPGGYGLPYT

SEQ ID NO: 5 - protein, 10 aa, synthetic construct
  MOD_RES 1: note = Biotinylated via an optional linker
LPGGYGLPYT

SEQ ID NO: 6 - protein, 10 aa, synthetic construct
PGGVPGGVFY

SEQ ID NO: 7 - protein, 11 aa, synthetic construct
  MOD_RES 11: note = Biotinylated
LPGGYGLPYTK

SEQ ID NO: 8 - protein, 11 aa, Mus musculus
HASQNINVWLS

SEQ ID NO: 9 - protein, 7 aa, Mus musculus
KASNLHT

SEQ ID NO: 10 - protein, 9 aa, Mus musculus
QQGQSYPLT

SEQ ID NO: 11 - protein, 5 aa, Mus musculus
DYYMS

SEQ ID NO: 12 - protein, 19 aa, Mus musculus
FIRNKANGYTTEYSASVKG

SEQ ID NO: 13 - protein, 11 aa, Mus musculus
DIITTPSWFGY

SEQ ID NO: 14 - protein, 74 aa, Mus musculus
HASQNINVWLSWYQQKPGNIPKLLIYKASNLHTGVPSRFSGSGSGTGFTLTISSLQPEDIATYYCQQGQSYPLT

SEQ ID NO: 15 - protein, 81 aa, Mus musculus
DYYMSWVRQPPGKALEWLGFIRNKANGYTTEYSASVKGRFTISRDNSQSILYLQMNTLRAEDSATYYCARDIITTPSWFGY

SEQ ID NO: 16 - protein, 107 aa, Mus musculus
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGAGTKLELK

SEQ ID NO: 17 - protein, 122 aa, Mus musculus
EVKLVESGGGLVQPGDSLRLSCATSGFTFTDYYMSWVRQPPGKALEWLGFIRNKANGYTTEYSASVKGRFTISRDNSQSILYLQMNTLRAEDSATYYCARDIITTPSWFGYWGQGTLVTVSA

SEQ ID NO: 18 - protein, 214 aa, Mus musculus
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 446 aa, Mus musculus
EVKLVESGGGLVQPGDSLRLSCATSGFTFTDYYMSWVRQPPGKALEWLGFIRNKANGYTTEYSASVKGRFTISRDNSQSILYLQMNTLRAEDSATYYCARDIITTPSWFGYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK